SEQUENCE LISTING (3 sequences)

SEQ ID NO: 1 - DNA, 1200 bp, Homo sapiens
  source 1..1200: mol_type = other DNA
  variation 346..348: note = D116N mutation
atggacagcagcgctgcccccacgaacgccagcaattgcactgatgccttggcgtactcaagttgctccccagcacccagccccggttcctgggtcaacttgtcccacttagatggcaacctgtccgacccatgcggtccgaaccgcaccgacctgggcgggagagacagcctgtgccctccgaccggcagtccctccatgatcacggccatcacgatcatggccctctactccatcgtgtgcgtggtggggctcttcggaaacttcctggtcatgtatgtgattgtcagatacaccaagatgaagactgccaccaacatctacattttcaaccttgctctggcaaatgccttagccaccagtaccctgcccttccagagtgtgaattacctaatgggaacatggccatttggaaccatcctttgcaagatagtgatctccatagattactataacatgttcaccagcatattcaccctctgcaccatgagtgttgatcgatacattgcagtctgccaccctgtcaaggccttagatttccgtactccccgaaatgccaaaattatcaatgtctgcaactggatcctctcttcagccattggtcttcctgtaatgttcatggctacaacaaaatacaggcaaggttccatagattgtacactaacattctctcatccaacctggtactgggaaaacctgctgaagatctgtgttttcatcttcgccttcattatgccagtgctcatcattaccgtgtgctatggactgatgatcttgcgcctcaagagtgtccgcatgctctctggctccaaagaaaaggacaggaatcttcgaaggatcaccaggatggtgctggtggtggtggctgtgttcatcgtctgctggactcccattcacatttacgtcatcattaaagccttggttacaatcccagaaactacgttccagactgtttcttggcacttctgcattgctctaggttacacaaacagctgcctcaacccagtcctttatgcatttctggatgaaaacttcaaacgatgcttcagagagttctgtatcccaacctcttccaacattgagcaacaaaactccactcgaattcgtcagaacactagagaccacccctccacggccaatacagtggatagaactaatcatcagctagaaaatctggaagcagaaactgctccgttgccc

SEQ ID NO: 2 - protein, 400 aa, Homo sapiens
  VARIANT 116: note = D116N mutation
MDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALANALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQLENLEAETAPLP

SEQ ID NO: 3 - DNA, 7138 bp, synthetic construct
  source 1..7138: mol_type = other DNA; note = pAAV-EF1a-FLEX-LAMuOR-P2A-nls-miRFP670nano
cgctcgctcactgaggccgcccgggcaaagcccgggcgtcgggcgacctttggtcgcccggcctcagtgagcgagcgagcgcgcagagagggagtggccaactccatcactaggggttcctgcggccgctcggtccgcacgtgagatctcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaagatctctcgagcagcgctcggtatcgatgcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacataggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatcgataagcttgatatcgaattcataacttcgtatagcatacattatacgaagttattctttgcaccattctaaagaataacagtgataatttctgggttaaggcaaataacttcgtataggatactttatacgaagttatgctagcagagaagttcctattcttataggagtataggaacttcgggcttaagcttggtaccaccatggacagcagcgctgcccccacgaacgccagcaattgcactgatgccttggcgtactcaagttgctccccagcacccagccccggttcctgggtcaacttgtcccacttagatggcaacctgtccgacccatgcggtccgaaccgcaccgacctgggcgggagagacagcctgtgccctccgaccggcagtccctccatgatcacggccatcacgatcatggccctctactccatcgtgtgcgtggtggggctcttcggaaacttcctggtcatgtatgtgattgtcagatacaccaagatgaagactgccaccaacatctacattttcaaccttgctctggcaaatgccttagccaccagtaccctgcccttccagagtgtgaattacctaatgggaacatggccatttggaaccatcctttgcaagatagtgatctccatagattactataacatgttcaccagcatattcaccctctgcaccatgagtgttgatcgatacattgcagtctgccaccctgtcaaggccttagatttccgtactccccgaaatgccaaaattatcaatgtctgcaactggatcctctcttcagccattggtcttcctgtaatgttcatggctacaacaaaatacaggcaaggttccatagattgtacactaacattctctcatccaacctggtactgggaaaacctgctgaagatctgtgttttcatcttcgccttcattatgccagtgctcatcattaccgtgtgctatggactgatgatcttgcgcctcaagagtgtccgcatgctctctggctccaaagaaaaggacaggaatcttcgaaggatcaccaggatggtgctggtggtggtggctgtgttcatcgtctgctggactcccattcacatttacgtcatcattaaagccttggttacaatcccagaaactacgttccagactgtttcttggcacttctgcattgctctaggttacacaaacagctgcctcaacccagtcctttatgcatttctggatgaaaacttcaaacgatgcttcagagagttctgtatcccaacctcttccaacattgagcaacaaaactccactcgaattcgtcagaacactagagaccacccctccacggccaatacagtggatagaactaatcatcagctagaaaatctggaagcagaaactgctccgttgccctccggagccactaacttctccctgttgaaacaagcaggggatgtcgaagagaatcccgggccatctagaatggcccccaagaagaagaggaaggtgatggcaaacctggacaagatgctgaataccacagtaacagaggtgcggcagttcctgcaggtggacagagtgtgcgtgttccagtttgaggaggattatagcggagtggtggtggtggaggccgtggacgataggtggatctccatcctgaagacccaggtgcgggatagatacttcatggagacaaggggcgaggagtattctcacggccgctaccaggccatcgccgacatctacaccgcaaacctgacagagtgctacagggatctgctgacacagtttcaggtgagagcaatcctggccgtgcccatcctgcagggcaagaagctgtggggcctgttggtggcacaccaactggcggcccctagacagtggcagacctgggagatcgactttctgaagcagcaggccgtggtggtgggcatcgccatccagcagagctaataagaagttcctattcttataggagtataggaacttcgggcgcgccataacttcgtataatgtatgctatacgaagttattaagaggtttcatattgctaatagcagctacaatccagctaccattctgcataacttcgtataaagtatcctatacgaagttattccggagtcgactctagaggatccggtacctcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccaattcccactcctttcaagacctagaaggtccattagctgcaaagattcctctctgtttaaaactttatccatctttgcaaagcttacgcgtgcggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgct

(19) United States
(12) Patent Application Publication
(10) Pub. No.: US 2025/0242058 A1
(43) Pub. Date: Jul. 31, 2025

(54) A GENE-BASED APPROACH TO CONFER LONG-LASTING PROTECTION FROM OPIOID USE DISORDER

(71) Applicant: Albert Einstein College of Medicine, Bronx, NY (US)

(72) Inventors: Lucas Sjulson, New York, NY (US); Kelly Clemenza, New York, NY (US)

(21) Appl. No.: 18/854,743

(22) PCT Filed: Apr. 14, 2023
(86) PCT No.: PCT/US2023/065796

§ 371 Date: Oct. 7, 2024

Related U.S. Application Data
(60) Provisional application No. 63/331,315, filed on Apr. 15, 2022.

(51) Int. Cl. A61K 48/00; A61K 38/17; A61P 25/36; C12N 5/0793; C12N 15/86

(52) U.S. Cl. CPC A61K 48/0058; A61K 38/1787; A61K 48/0083; A61P 25/36; C12N 5/0619; C12N 15/86; C12N 2750/14143; C12N 2800/30; C12N 2830/008

(57) ABSTRACT

A composition and method for treatment of opioid use disorder (OUD) is provided by expressing a novel mu receptor mutant, LAMuOR (Low Affinity Mu Opioid Receptor), with reduced binding affinity for opioids such that it is activated only by the high concentration of exogenous opioids encountered in OUD but not by the low concentrations of endogenous opioids that occur physiologically. When expressed in specific brain circuits, these low-affinity mu opioid receptors can suppress reward-related neuronal activity specifically in the presence of opioids of abuse, thereby reducing abuse potential.

### CROSS-REFERENCE TO RELATED APPLICATIONS

This application claims priority to U.S. Provisional Application 63/331,315 filed on Apr. 15, 2022, which is incorporated herein by reference in its entirety.

### BACKGROUND

Substance use disorders are characterized by progressively uncontrollable substance use that persists in spite of negative consequences (e.g., social, economic and/or medical consequences). Substance use disorders are marked by a transition from substance use that is well controlled, to a use that is unregulated and destructive. This transition can be abrupt or progressive in nature. Substance use disorders are characterized by addiction, or dependence, upon the substance. When a subject is dependent upon, or addicted to, a substance, this means that there is a physical, physiological or psychological reaction and/or interaction of the substance and the subject, which results in the subject exhibiting or having a forced or compulsive use of the substance without a recognized purpose or need for treating a disease. Rather, the purpose is that of achieving the desired effect, and/or avoiding withdrawal symptoms as defined hereinafter, which occur when the substance is discontinued or the amount used is reduced. Substance use disorders are sometimes referred to as “substance abuse”, and the substance or substances to which the subject is addicted or dependent upon are “abused”, for example opioid abuse or alcohol abuse.

One particularly acute set of substance use disorders are opioid-based substance use disorders. There is a devastating opioid use and overdose crisis in the United States. It has been reported that approximately 25.5 million adults suffer pain and opioids that are often prescribed for their treatment can lead to opioid misuse and dependence. It has also been reported that more than 2 million Americans have opioid use disorders (OUDs), and many started their addiction with prescribed opioids. One of the most devastating consequences of opioid misuse is opioid overdose, which can produce respiratory depression and death. Drug overdose is the leading cause of accidental death in the US, with an estimation of 60,000 deaths in 2016.

Although there are safe and effective pharmacotherapies for opioid use disorders and to prevent/reverse overdose, their use has some limitation, or they are largely underutilized. Methadone and buprenorphine are approved by the FDA to treat opioid use disorders (OUDs) but long term efficacy and treatment adherence (e.g., visiting a methadone clinic seven days a week) are sub-optimal. Naltrexone is approved by the FDA to prevent opioid use relapse, but initiation and adherence to treatment are low, and it may increase the long-term risk of overdose. Naloxone (Narcan) is approved to reverse opioid overdose, but it does not treat chronic OUD. Lofexidine is the only non-opioid drug to be FDA approved for helping mitigate opioid withdrawal symptoms, and clonidine is also used off-label for this purpose. However, these are only useful during acute withdrawal and do not prevent relapse. Other medications such as gabapentin, pregabalin, buprenorphine, cannabinoids, ketamine, and ultra-low doses of oral naltrexone, as well as rapid opioid withdrawal under anesthesia have been evaluated but the results are inconclusive.

There thus exists an unmet and pressing need for novel methods to treat substance use disorders.

### BRIEF SUMMARY

In an aspect, a method of treating a subject with a substance use disorder comprises administering to the subject a composition comprising a vector comprising an expression cassette. The expression cassette comprises a nucleic acid encoding a low affinity human Mu opioid receptor (LAMuOR) expressible in a neuronal cell. Upon administering, the LAMuOR is expressed in DIR-positive direct pathway medium spiny neurons of the striatum of the subject in an amount effective to reduce direct pathway activity, and/or is expressed in dopaminergic neurons in the ventral tegmental area of the subject in an amount effective to reduce dopamine release, such that exogenous opioid-induced dopamine release is reduced without affecting release of endogenous opioid-induced dopamine. The administering reduces at least one symptom of the substance use disorder, or prevents a relapse of the substance use disorder, reduces tolerance to or dependence on a substance of the substance abuse disorder, improves adherence of a treatment for a substance use disorder, reduces preference or decreases liking, or improves abstinence of a substance of the substance use disorder, in the subject. In one aspect the substance use disorder is an opioid use disorder.

In another aspect, an expression cassette comprises a nucleic acid encoding a low affinity Mu opioid receptor (LAMuOR) expressible in a neuronal cell. In yet another aspect, a vector comprises the expression cassette. In yet another aspect, a neuronal cell is transduced with the vector comprising the expression cassette.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-B show a schematic of the rationale underlying LAMuOR (Low-Affinity Mu Opioid Receptor) as a tool to prevent opioid drugs from stimulating dopamine release in the NAc. Up arrows indicate increased activity, and down arrows indicate reduced activity. Stippling represents activation. (1A) Opioids of abuse, such as fentanyl or heroin, stimulate dopamine release indirectly by activating MuORs on GABAergic cells in the ventral tegmental area (VTA). MuOR activation suppresses GABA release, which allows the VTA dopaminergic cells to become overactive and release supraphysiological amounts of dopamine in the nucleus accumbens (NAc). Increased dopamine release in the NAc is believed to be the “final common pathway” of addictive drugs. (1B) The LAMuOR strategy counteracts this process by using adeno-associated viral vectors (AAVs) to express the human MuOR D114N mutant (human LAMuOR-D114N, rat protein numbering), ectopically in dopaminergic VTA cells. As used herein, particularly in the Examples, the convention D114N is used for the human LAMuOR D116N mutation. The D114N is an older convention based on the numbering of the rat mutation. The D114N mutation is equivalent to D116N in the human sequence. Under baseline conditions, the low affinity of the receptor prevents activation by endogenous opioids. However, the high concentrations of opioids present during opioid abuse activate LAMuOR and suppress dopamine (DA) release.

FIGS. 2A-D show LAMuOR-D114N suppresses fentanyl-induced NAc dopamine release but leaves cocaine-induced dopamine elevation intact. (2A) Diagram of the experiment expressing LAMuORD114N unilaterally in dopaminergic cells of the ventral tegmental area (VTA) and measuring dopamine signals in the nucleus accumbens (NAc). (2B) Mice expressing LAMuOR-D114N unilaterally exhibit a striking rotation behavior after receiving fentanyl. This behavior is known to be associated with unilateral suppression of dopamine release. (2C-D) Fiber photometric recordings of dopamine levels show that LAMuOR suppresses fentanyl-induced dopamine release but leaves cocaine-induced dopamine elevation intact.

FIGS. 3A-G show LAMuOR-D114N reduces fentanyl self-administration without affecting other behaviors in the absence of fentanyl. (3A) Diagram of the experiment expressing LAMuOR-D114N bilaterally in dopaminergic cells of the ventral tegmental area (VTA). (3B) LAMuOR-D114N does not affect open field locomotion in the absence of fentanyl, but it blocks fentanyl-induced hyperlocomotion. At the highest AAV-LAMuOR dose, fentanyl actually reduces locomotion, suggesting that dopamine levels are suppressed below baseline. (3C) Group statistics summarizing results from 3B. (3D) LAMuOR-D114N does not affect cocaine-induced hyperlocomotion, suggesting the effect is specific for opioids. (3E) A two-bottle choice assay used to test preference for oxycodone or sucrose solution. (3F) LAMuOR-D114N reduces oral oxycodone self-administration in a two-bottle choice model. (3G) LAMuOR-D114N does not reduce sucrose preference in two-bottle choice, suggesting it does not affect physiological reward-seeking behaviors.

FIG. 4 is a schematic of pAAV-EF1a-FLEX-LAMuOR-P2A-nls-miRFP670nano. Nucleic acid sequence of the vector is shown in SEQ ID NO:3.

The above-described and other features will be appreciated and understood by those skilled in the art from the following detailed description, drawings, and appended claims.

### DETAILED DESCRIPTION

The disclosure provides compositions and methods of treatment that can meet this need for novel methods to treat substance use disorders and further address the need for a treatment by which a single procedure could confer lifelong protection against OUD without requiring adherence to any regimen.

The rewarding and reinforcing effects of addictive opioids are mediated by activation of the mu opioid receptor (MuOR), a Gi-coupled G-protein coupled receptor that exerts an overall inhibitory effect on MuOR-expressing cells. MuOR is expressed in many cell populations throughout the brain, but a major contributor to the addictive potential of opioids is MuOR expression in GABAergic neurons of the ventral tegmental area (VTA). In the absence of opioids, these GABAergic neurons inhibit the dopaminergic VTA neurons that project to the nucleus accumbens (NAc).

Activation of these MuORs by opioids inhibits the GABAergic neurons, thereby releasing the dopaminergic cells from inhibition and increasing dopamine levels in the NAc. Increasing dopamine levels in the NAc causes euphoria, the final pathway of all forms of drug addiction, including OUD.

The inventors have discovered that administering an expression cassette encoding a low affinity MuOR (LAMuOR) to a subject in need thereof can suppress exogenous opioid-induced dopamine release without affecting endogenous opioid-induced dopamine release, thereby reducing drug dependence.

Finding a means to suppress or counteract the opioid-induced dopamine release could provide a powerful therapeutic strategy for OUD. However, MuOR antagonists paradoxically increase the risk of fatal overdose upon relapse, and systemic dopamine receptor antagonists would cause too many side effects to be a viable therapeutic strategy. One method for targeting the dopaminergic VTA cells with high specificity is chemogenetics, which refers to using gene therapy methods to express mutant neurotransmitter receptors in specific brain circuits. These receptors contain mutations that prevent them from being activated by endogenous ligands, and they are instead activated by specialized drug-like compounds designed to have minimal off-target effects. The most common chemogenetic receptors are called DREADDs (Designer Receptors Exclusively Activated by Designer Drugs), and the inhibitory DREADD hM4Di has been used successfully in mice to suppress VTA dopamine release, stopping heroin self-administration. However, DREADDs have two serious problems: 1) dopamine release would be suppressed all the time, not just in the presence of opioids. This would likely cause unacceptable neuropsychiatric side effects and affect physiological reward-related behaviors. 2) DREADDs will fail unless the patient adheres stringently to a daily medication regimen.

The inventors have discovered a novel approach using Low-Affinity Mu Opioid Receptors (LAMuOR) to specifically suppress opioid-induced VTA dopamine release. These mutant LAMuORs have reduced binding affinity for opioids, so they are activated only by the high concentrations of exogenous opioids encountered in OUD but not by the low concentrations of endogenous opioids that occur physiologically. Hence, when expressed in specific brain circuits, these LAMuORs can suppress reward-related neuronal activity specifically in the presence of opioids of abuse, thereby reducing their reward value and abuse potential. The data demonstrate that expression of a specific LAMuOR mutant (LAMuOR-D114N: rat numbering, equivalent to human LAMuOR-D116N) in neuronal cells in the VTA effectively reduces behavioral and physiological responses to fentanyl in mice such as opioid seeking, with no observed behavioral or physiological side effects in the absence of fentanyl. Without being bound to a theory, the LAMuOR exerts an inhibitory effect through Gi activation, but it is activated by exogenous opioids rather than a traditional DREADD ligand. This avoids the problems associated with traditional DREADDs yet would confer a lifelong therapeutic effect from a single treatment, a distinct advantage over existing OUD treatments.

### The Opioid System

Opioid receptors. The opioid system consists of four G protein-coupled receptors, mu, delta, kappa and opioid receptor-like 1, which are stimulated by a family of endogenous opioid peptides. Opioid receptors can also be activated exogenously by alkaloid opiates and synthetic fentanyls.

Opioid receptors are located both in the central and peripheral nervous system. Mu opioid receptors are expressed in many cell populations throughout the brain. Mu opioid receptors consist of a single polypeptide chain possessing an extracellular N-terminal region, seven transmembrane domains and an intracellular C-terminal tail. MORs are generally found postsynaptically on dendrites and cell bodies, on glutamatergic and GABAergic axon terminals and postsynaptically on cells expressing dopamine receptors.

The mu opioid receptor is located in the nucleus accumbens, ventral tegmental area (VTA), and cortex and is responsible for the analgesic, rewarding and unwanted effects of opioids and similar drugs. Activation of the mu receptor by ligands induces significant changes inside the cell, such as an inhibition of adenylate cyclase activity, activation of potassium channels and reductions of calcium conductance among other signaling pathways such as phospholipase C, mitogen-activated kinases (MAP kinases) or beta-arrestin (involved in receptor desensitization and internalization/recycling) involved in opioid activity.

The connections within the limbic subcircuit of striatal circuitry including GABAergic neurons of the nucleus accumbens (NAc), dopaminergic neurons of the VTA, and glutamatergic neurons of the prefrontal cortex (PFC) are known to contribute to opioid use disorders, but the major contributor to the addictive potential of opioids is MuOR expression in GABAergic neurons of the VTA. In the absence of opioids, these GABAergic neurons inhibit dopaminergic VTA neurons that project into the NAc.

Dopamine receptors. Dopamine is a neurotransmitter produced in neuronal terminals and loaded into synaptic vesicles. Dopamine transmission is tightly controlled at the presynaptic level.

There are five types of dopamine receptors D1-D5, a superfamily of G protein-coupled receptors grouped into two categories, D1-like (D1, D5) and D2-like (D2, D3, D4) receptors, based on the functional properties to stimulate adenylyl cyclase (AC) via Gs/olf and to inhibit AC via Gi/o, respectively. In the striatum, expression of excitatory D1 type and inhibitory D2 type receptor is segregated in two types of GABAergic medium spiny neurons, striatonigral/direct and striatopallidal/indirect pathway neurons, respectively. The striatonigral/direct pathway neurons contain the excitatory D1 type dopamine receptors (e.g., DIR-positive medium spiny neurons of the direct pathway) which respond mostly to dopamine burst signals. Dopamine neurons project in the ventral tegmental area (VTA) of the midbrain to the ventral striatum, in particular the nucleus accumbens (NAc) core and shell, and to other frontal targets including the prefrontal cortex.

Endogenously, opioid receptors are stimulated by endogenous peptides, such as endomorphins, dynorphins and enkephalins. Endomorphin 1 and endomorphin-2 have the highest affinity and selectivity for the mu-opioid receptor in the central and peripheral nervous systems.

Binding of an endogenous (endorphin molecule) or exogenous (morphine molecule or other opioid drug such as codeine, fentanyl, or buprenorphine) ligand with an opioid receptor leads to activation of a Go or Gi protein and to subsequent phosphorylation by a family of kinases called the G protein-coupled receptor kinases. This induces molecular changes inside the cell, including β-arrestin binding. Chronic exposure to exogenous opioids induces the phosphorylation of opioid receptors which prepares opioid receptors for β-arrestin binding thereby blocking further G-protein mediated signaling, thereby desensitizing the opioid receptors.

In some aspects of the disclosure, the opioid comprises heroin, codeine, fentanyl, hydrocodone (dihydrocodeinone), hydromorphone, meperidine, methadone, morphine, oxycodone, oxymorphone, and combinations thereof. A non-limiting list of opioid drugs which can cause OUD are listed in Table 1.

TABLE 1
OPIOIDS

| Name | Brand Name ® | Additional names/information |
|---|---|---|
| heroin | | from poppy, papaver |
| | | somniferum; aka diamorphine |
| mitragynine | | from Mitragyna speciosa |
| codeine | | (5α,6α)-7,8-didehydro-4,5-epoxy- |
| | | 3-methoxy-17-methylmorphinan- |
| | | 6-ol |
| fentanyl | Actiq ®, | |
| | Duragesic ®, | |
| | Sublimaze ®, | |
| | Subsys ®, | |
| | Abstral ®, Ionsys ® | |
| hydrocodone | Vicodin ®, | |
| | Norco ®, | |
| | Zohydro ® | |
| dihydrocodeinone | Lorcet ®, Hycet ®, | |
| | Zamicet ®, Xodol ® | |
| hydromorphone | Dilaudid ®, | |
| | Exalgo ® ER | |
| meperidine | Demerol ® | |
| methadone | Dolophine ®, | |
| | Methadose ™ | |
| morphine | Duramorph ®, MS | |
| | Contin ®, | |
| | Infumorph ® P/F, | |
| | Arymo ®, | |
| | Astramorph-PF ™ | |
| oxycodone | Oxycontin ®, | |
| | Roxicodone ®, | |
| | Percodan ®, | |
| | Percocet ® | |
| oxymorphone | Opana ® | |
| remifentanil | Ultiva ® | methyl 1-(2-methoxycarbonylethyl)-4-(phenyl- |
| | | propanoyl-amino)-piperidine-4-carboxylate |
| carfentanil | Wildnil ™ | (4-methoxycarbonyl)fentanyl |
| sufentanil | Dsuvia ®, | N-[4-(methoxymethyl)-1-(2-thiophen-2- |
| | Zalviso ™ | ylethyl)piperidin-4-yl]-N-phenylpropanamide |
| buprenorphine | Buprenex ®, | (1S,2S,6R,14R,15R,16R)-5-(cyclopropylmethyl)- |
| | Temgesic ™ | 16-[(2S)-2-hydroxy-3,3-dimethylbutan-2-yl]-15- |
| | | methoxy-13-oxa-5- |
| | | azahexacyclo[13.2.2.1^{2,8}.0^{1,6}.0^{2,14}.0^{12,20}]icosa- |
| | | 8(20),9,11-trien-11-ol |

In an aspect, an expression cassette comprising a nucleic acid sequence encoding a Mu opioid receptor with a mutation in one or more residues of the human MuOR (SEQ ID NO:2) at position 116(2.50), 149(3.32), 331(7.46), or 334(7.49). Equivalent residues in the rat MuOR are 114(2.50), 147(3.32), 329(7.46), or 332(7.49). The numbers in parentheses represent the residue numbers using the Ballesteros-Weinstein numbering system. The mutation can be any change that would produce a low affinity mu opioid receptor with reduced affinity to a mu receptor agonist with reduced activation by endogenous opioid peptides. In one aspect, the mutation is a D116N mutation (LAMuOR-D116N). In one aspect, the MuOR is a human MuOR (GenBank accession NM_000914.5 and NP_000905.3). In another aspect, the MuOR is a human MuOR containing a D116N mutation (nucleic acid sequence identified SEQ ID NO:1, and amino acid sequence identified in SEQ ID NO:2), or a variant thereof, which preferentially reduces affinity for endogenous opioids but leaves affinity for exogenous opioids relatively intact. Asp116 (human) or Asp114 (rat) in the second transmembrane region of the mu receptor is critical for agonist binding. Mutation of this charged residue to a neutral asparagine results in a mutant receptor that exhibits greatly reduced affinity for endogenous opioid peptides. In contrast, antagonists such as naloxone bind with high affinity to this mutant.

As used herein, the term “variant” refers to a polynucleotide or polypeptide having a sequence substantially similar to a reference polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

The expression cassette can be operably linked to an expression control element. The term “element” refers to a separate or distinct part of something, for example, a nucleic acid sequence with a separate function within a longer nucleic acid sequence. The term “regulatory element” and “expression control element” are used interchangeably herein and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements. Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

In a specific aspect, the expression cassette is in a vehicle for expression in a neuronal cell. In an aspect the neuronal cell is in the brain of a subject. In a specific aspect, the vehicle is a vector for carrying and transferring a nucleic acid to a neuronal cell. Non-limiting examples of vectors include plasmids and viral vectors (for example, AAV, lentivirus, herpes simplex virus vectors). In one aspect, such vectors are rAAV vectors. It will be appreciated that other cloning vectors may be used in the invention, and therefore reference to AAV herein may be taken to refer to any suitable vector.

The term “AAV” or “adeno-associated virus” refers to a Dependoparvovirus within the Parvoviridae genus of viruses. For example, the AAV can be an AAV derived from a naturally occurring “wild-type” virus, an AAV derived from a rAAV genome packaged into a capsid derived from capsid proteins encoded by a naturally occurring cap gene and/or a rAAV genome packaged into a capsid derived from capsid proteins encoded by a non-natural capsid cap gene.

The term “rAAV” refers to a “recombinant AAV”. In some embodiments, a recombinant AAV has an AAV genome in which part or all of the rep and cap genes have been replaced with heterologous sequences.

The term “cap gene” refers to the nucleic acid sequences that encode capsid proteins that form, or contribute to the formation of, the capsid, or protein shell, of the virus. In the case of AAV, the capsid protein may be VP1, VP2, or VP3. For other parvoviruses, the names and numbers of the capsid proteins can differ.

The term “rep gene” refers to the nucleic acid sequences that encode the non-structural proteins (rep78, rep68, rep52 and rep40) required for the replication and production of virus.

AAV vectors that comprise coding regions of one or more LAMuORs of interest are provided. The AAV vector can include a 5′ inverted terminal repeat (ITR) of AAV, a 3′ AAV ITR, a promoter driving expression of the LAMuOR nucleic acid, and a restriction site downstream of the promoter to allow insertion of a polynucleotide encoding one or more LAMuORs of interest, wherein the promoter and the restriction site are located downstream of the 5′ AAV ITR and upstream of the 3′ AAV ITR. In some embodiments, the AAV vector includes a posttranscriptional regulatory element, for example the Woodchuck Hepatitis Virus Posttranscriptional Regulatory Element (WPRE), downstream of the restriction site and upstream of the 3′ AAV ITR. In some embodiments, the AAV vectors disclosed herein can be used as AAV transfer vectors carrying a transgene encoding a protein of interest for producing recombinant AAV viruses that can express the protein of interest in a host cell.

The nucleotide sequences of AAV ITR regions are known. The ITR sequences for AAV-2 are described in the art. The skilled artisan will appreciate that AAV ITR's can be modified using standard molecular biology techniques. Accordingly, AAV ITRs used in the vectors of the invention need not have a wild-type nucleotide sequence, and may be altered, e.g., by the insertion, deletion or substitution of nucleotides. Additionally, AAV ITRs may be derived from any of several AAV serotypes, including but not limited to, AAV-1, AAV-2, AAV-3, AAV-4, AAV-5, AAVX7, and the like. Furthermore, 5′ and 3′ ITRs which flank a selected nucleotide sequence in an AAV expression vector need not necessarily be identical or derived from the same AAV serotype or isolate, so long as the ITR's function as intended, i.e., to allow for excision and replication of the bounded nucleotide sequence of interest when AAV rep gene products are present in the cell.

rAAV vectors are provided herein. Generation of the viral vector can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)).

In some embodiments, the viral vectors can include additional sequences that make the vectors suitable for replication and integration in eukaryotes. In other embodiments, the viral vectors disclosed herein can include a shuttle element that makes the vectors suitable for replication and integration in both prokaryotes and eukaryotes. In some embodiments, the viral vectors can include additional transcription and translation initiation sequences, such as promoters and enhancers; and additional transcription and translation terminators, such as polyadenylation signals. In some embodiments, the vector can also comprise regulatory control elements known to one of skill in the art to influence the expression of the RNA and/or protein products encoded by the polynucleotide within desired cells of the subject.

Expression control elements and promoters include those active in a particular tissue or cell type, referred to herein as a “tissue-specific expression control elements/promoters.” Tissue-specific expression control elements are typically active in specific cell or tissue (for example in the brain, central nervous system, spinal cord, peripheral nervous system, retina or lung). Expression control elements are typically active in these cells, tissues or organs because they are recognized by transcriptional activator proteins, or other regulators of transcription, that are unique to a specific cell, tissue or organ type.

Expression control elements also include ubiquitous or promiscuous promoters/enhancers which are capable of driving expression of a polynucleotide in many different cell types. Such elements include, but are not limited to, the cytomegalovirus (CMV) immediate early promoter/enhancer sequences, the Rous sarcoma virus (RSV) promoter/enhancer sequences and the other viral promoters/enhancers active in a variety of mammalian cell types; promoter/enhancer sequences from ubiquitously or promiscuously expressed mammalian genes including, but not limited to, beta actin, ubiquitin or EF1alpha; or synthetic elements that are not present in nature.

Alternatively, the regulatory sequences of the AAV vector can direct expression of the gene preferentially in a particular cell type, i.e., tissue-specific regulatory elements can be used. Non-limiting examples of tissue-specific promoters that can be used include, central nervous system (CNS) specific promoters such as, neuron-specific promoters (e.g., the human synapsin promoter). Preferably, the promoter is tissue specific and is essentially not active outside the central nervous system, or the activity of the promoter is higher in the central nervous system that in other systems. For example, a promoter specific for the spinal cord, brainstem, (medulla, pons, and midbrain), cerebellum, diencephalon (thalamus, hypothalamus), telencephalon (corpus striatum, cerebral cortex, or within the cortex, the occipital, temporal, parietal or frontal lobes), or combinations, thereof. The promoter may be specific for particular cell types, such as neurons in the CNS. If it is active in neurons, it may be specific for particular types of neurons, e.g., motor neurons, sensory neurons, or interneurons. Preferably, the promoter is specific for cells in particular regions of the brain, for example, the cortex, striatum, substantia nigra, and hippocampus. In an aspect, the promoter is specific for dopaminergic neurons or direct pathway medium spiny neurons in the striatum.

Exemplary neuronal specific promoters include, but are not limited to, neuron specific enolase (NSE), (GenBank Accession No: X51956), and human neurofilament light chain promoter (NEFL) (GenBank Accession No: L04147). In a preferred embodiment, the gene of interest is flanked upstream (i.e., 5′) by the elongation factor 1 alpha (EF) promoter.

Expression control elements also can confer expression in a manner that is regulatable, that is, a signal or stimuli increases or decreases expression of the operably linked polynucleotide. A regulatable element that increases expression of the operably linked polynucleotide in response to a signal or stimuli is also referred to as an “inducible element” (that is, it is induced by a signal). Particular examples include, but are not limited to, a hormone (for example, steroid) inducible promoter. A regulatable element that decreases expression of the operably linked polynucleotide in response to a signal or stimuli is referred to as a “repressible element” (that is, the signal decreases expression such that when the signal, is removed or absent, expression is increased). Typically, the amount of increase or decrease conferred by such elements is proportional to the amount of signal or stimuli present; the greater the amount of signal or stimuli, the greater the increase or decrease in expression.

In one aspect, a vector comprises an insert, preferably comprising a nucleic acid sequence encoding the LAMuOR gene or a variant thereof with flanking DNA. The flanking DNA may comprise one or more selectable markers or one or more site specific recombination sites. In one aspect the vector comprises 2 or more, such as 3, heterospecific and incompatible site-specific recombination sites. In one aspect the site specific recombination sites may be loxP sites, or variants thereof, or FRT sites or variants thereof. Lox sites are directional 35 bp sequences that can flank the LAMuOR transgene sequence and are located between the ITRs. Lox sites, in combination with the Cre recombinase, are used to spatially and temporally control gene expression. Transfer plasmids with two pairs of lox sites are part of a dual system called FLEX or DIO. When multiple lox sites are used, Cre recombinase first flips the transgene sequence between the first pair of lox sites, and then excises the sequence between the second pair.

The Cre-LoxP system is a site-specific recombination system. Cre recombinase catalyzes site-specific recombination between two LoxP sites, (34 bp DNA fragment containing an asymmetric 8 bp spacer flanked by two 13 bp inverted repeats). The orientation of the two LoxP sites determines the outcome of the Cre-mediated recombination. When the two LoxP sites are arranged in same orientation, the recombinase can excise any intervening sequence flanked by LoxP sites. When the two LoxP sites are placed in the reciprocal orientation, the recombinase can inverse the sequence between the two LoxP sites.

Lox2272 site is a mutant of LoxP site carrying a mutation within the 8 bp spacer region of the LoxP site preventing these sites from recombining with wt LoxP site but can readily recombine with themselves. The FLEX switch allows two pairs of mismatched LoxP sites to first invert and turn on the switch and a subsequent excision event to eliminate on the LoxP partners to prevent re-inversion.

In one aspect, a cassette for expressing mutants of mu opioid receptor in neurons, the segments comprising a nucleic acid encoding a low affinity mu opioid receptor gene or a variant thereof, wherein the virus's single stranded DNA genome is processed into a double-stranded circular episome by the host cell's replication machinery. The episome is maintained extrachromosomally and can persist in non-dividing cells for years, allowing for long-term transgene expression in non-dividing cells.

In another aspect, the gene of interest is flanked by FRT sites or variants thereof to control for unwanted side effects. When the gene of interest is flanked by FRT sites, Flp recombinase, for example introduced in an additional rAAV encoding a Flp recombinase, can be introduced into cells containing the gene of interest such that the coding sequence is excised in the presence of Flp, and then is not in line with the promoter for expression, thereby silencing expression of the gene of interest.

Most AAV serotypes have the capability to transduce neurons, albeit with varying strength. For example, AAVs 1, 2, 5, 7, 8 and 9 all show strong preference for transducing neurons in vivo following brain injections. Serotype 1 (AAV2) has a natural tropism for neurons and is the most commonly used and characterized serotype. AAV tropism can also be further altered by creating recombinant versions of multiple AAV serotypes, a process known as pseudotyping. These pseudotyped viruses can have enhanced tropism for specific cell types, as well as improved transduction efficiency in neurons. Pseudotyping involves engineering new viral capsids from different serotypes to create rAAVs with different cell-type expression, for example expression in striatum, hippocampus, cortex, neurons, astrocytes, microglia.

AAV variants and rAAVs with different capabilities for targeting different cells in the nervous system, e.g., AAV-PHP.eB and AAV-PHP.S can target neurons in the CNS and PNS, respectively, when injected intravenously, bypassing the need to perform site-directed injections in the brain, are known in the art.

In order to produce recombinant AAV particles, an AAV vector can be introduced into a suitable host cell using known techniques, such as by transfection. A number of transfection techniques are generally known in the art. Exemplary transfection methods include calcium phosphate co-precipitation, direct micro-injection into cultured cells, electroporation, liposome mediated gene transfer, lipid-mediated transduction, and nucleic acid delivery using high-velocity microprojectiles.

Exemplary host cells for producing recombinant AAV particles include, but are not limited to, microorganisms, yeast cells, insect cells, and mammalian cells, that can be, or have been, used as recipients of an exogenous nucleic acid molecule. Thus, a “host cell” as used herein generally refers to a cell which has been transfected with an exogenous nucleic acid molecule. The host cell includes any eukaryotic cell or cell line so long as the cell or cell line is not incompatible with the protein to be expressed, the selection system chosen or the fermentation system employed. Non-limiting examples include CHO dhfr-cells, 293 cells (Graham et al. (1977) J. Gen. Virol. 36:59) or myeloma cells like SP2 or NSO.

Host cells containing the above-described AAV vectors must be rendered capable of providing AAV helper functions in order to replicate and encapsidate the expression cassette flanked by the AAV ITRs to produce recombinant AAV particles. AAV helper functions are generally AAV-derived coding sequences which can be expressed to provide AAV gene products that, in turn, function in trans for productive AAV replication. AAV helper functions are used herein to complement necessary AAV functions that are missing from the AAV vectors. Thus, AAV helper functions include one, or both of the major AAV open reading frames (ORFs), namely the rep and cap coding regions, or functional homologues thereof.

Alternatively, a vector of the invention can be a virus other than the adeno-associated virus, or portion thereof, which allows for expression of a nucleic acid molecule introduced into the viral nucleic acid. For example, replication defective retroviruses, adenoviruses and lentivirus can be used. Protocols for producing recombinant retroviruses and for infecting cells in vitro or in vivo with such viruses can be found in standard laboratory manuals. Examples of retroviruses include pLJ, pZIP, pWE and pEM which are well known to those skilled in the art. Examples of suitable packaging virus lines include Crip, Cre, 2 and Am. The genome of adenovirus can be manipulated such that it encodes and expresses the protein of interest but is inactivated in terms of its ability to replicate in a normal lytic viral life cycle. Exemplary adenoviral vectors derived from the adenovirus strain Ad type 5 dl324 or other strains of adenovirus (e.g., Ad2, Ad3, Ad7 etc.) are well known to those skilled in the art.

Alternatively, the vector can be delivered using a non-viral delivery system. This includes delivery of the vector to the desired tissues in colloidal dispersion systems that include, for example, macromolecule complexes, nanocapsules, microspheres, beads, and lipid-based systems including oil-in-water emulsions, micelles, mixed micelles, and liposomes.

Liposomes are artificial membrane vesicles which are useful as delivery vehicles in vitro and in vivo. In order for a liposome to be an efficient gene transfer vehicle, the following characteristics should be present: (1) encapsulation of the genetic material at high efficiency while not compromising the biological activity; (2) preferential and substantial binding to a target cell in comparison to non-target cells; (3) delivery of the aqueous contents of the vesicle to the target cell cytoplasm at high efficiency; and (4) accurate and effective expression of genetic information. Examples of lipids for liposome production include phosphatidyl compounds, such as phosphatidylglycerol, phosphatidylcholine, phosphatidylserine, phosphatidylethanolamine, sphingolipids, cerebrosides, and gangliosides. Additional examples of lipids include, but are not limited to, polylysine, protamine, sulfate and 3β-[N—(N′,N′ dimethylaminoethane) carbamoyl] cholesterol.

Alternatively, the vector can be coupled with a carrier for delivery. Exemplary and preferred carriers are keyhole limpet hemocyanin (KLH) and human serum albumin. Other carriers may include a variety of lymphokines and adjuvants such as INF, IL-2, IL-4, IL-8 and others. The vector can be conjugated to a carrier by genetic engineering techniques that are well known in the art. Also within the scope of the invention is the delivery of the vector in one or more combinations of the above delivery methods.

Also disclosed herein are pharmaceutical compositions comprising one or more of the rAAV viruses disclosed herein and one or more pharmaceutically acceptable carriers. The compositions can also comprise additional ingredients such as diluents, stabilizers, excipients, and adjuvants. As used herein, “pharmaceutically acceptable” carriers, excipients, diluents, adjuvants, or stabilizers are the ones nontoxic to the cell or subject being exposed thereto (preferably inert) at the dosages and concentrations employed or that have an acceptable level of toxicity as determined by the skilled practitioners.

The carriers, diluents and adjuvants can include buffers such as phosphate, citrate, or other organic acids; antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

Titers of the rAAV to be administered will vary depending, for example, on the particular rAAV, the mode of administration, the treatment goal, the individual, and the cell type(s) being targeted, and can be determined by methods standard in the art.

As will be readily apparent to one skilled in the art, the useful in vivo dosage of the recombinant virus to be administered and the particular mode of administration will vary depending upon the age, weight, the severity of the affliction, and animal species treated, the particular recombinant virus expressing the protein of interest that is used, and the specific use for which the recombinant virus is employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

Although the exact dosage will be determined on a drug-by-drug basis, in most cases, some generalizations regarding the dosage can be made. In some instances, the rAAV for delivery a nucleic acid to the nervous system (e.g., CNS) of a subject can be administered, for example via injection, to a subject at a dose of between 1×10⁶ genome copies (GC) of the recombinant virus and 2×10¹⁰ GC, for example between 5×10⁷ GC and 5×10¹² GC. In some instances, the dose of the rAAV administered to the subject is no more than 2×10¹⁰ GC. In some instances, the dose of the rAAV administered to the subject is no more than 5×10¹² GC. In some instances, the dose of the rAAV administered to the subject is no more than 5×10¹¹ GC.

The vectors disclosed herein can be administered to a subject (e.g., a human) in need thereof. The disclosure provides a method of treating opioid use disorder, comprising administering to the subject a therapeutically effective amount of a composition comprising the vector or a pharmaceutically acceptable salt thereof. In some embodiments of the methods of the disclosure, administering the composition reduces a symptom of withdrawal or prevents a relapse of the opioid use disorder in the subject.

Delivery systems include methods of in vitro, in vivo and ex vivo delivery of the vector. For in vivo delivery, the vector can be administered to a subject in a pharmaceutically acceptable carrier. The term “pharmaceutically acceptable carrier”, as used herein, refers to any physiologically acceptable carrier for in vivo administration of the vectors of the present invention. Such carriers do not induce an immune response harmful to the individual receiving the composition and are discussed above. In one embodiment, vector can be distributed throughout a wide region of the CNS, by injecting the vector into the cerebrospinal fluid, e.g., by lumbar puncture.

Alternatively, precise delivery of the vector into specific sites of the brain, can be conducted using stereotactic microinjection techniques. For example, the subject being treated can be placed within a stereotactic frame base (MRI-compatible) and then imaged using high resolution MRI to determine the three-dimensional positioning of the particular region to be treated. The MRI images can then be transferred to a computer having the appropriate stereotactic software, and a number of images are used to determine a target site and trajectory for antibody microinjection. The software translates the trajectory into three-dimensional coordinates that are precisely registered for the stereotactic frame. In the case of intracranial delivery, the skull will be exposed, burr holes will be drilled above the entry site, and the stereotactic apparatus used to position the needle and ensure implantation at a predetermined depth. The vector can be delivered to regions, such as the cells of the spinal cord, brainstem, (medulla, pons, and midbrain), cerebellum, diencephalon (thalamus, hypothalamus), telencephalon (corpus striatum, cerebral cortex, or within the cortex, the occipital, temporal, parietal or frontal lobes), or combinations, thereof. In another preferred embodiment, the vector is delivered via intravascular approaches in combination with approaches for disruption of the blood-brain barrier, such as focused ultrasound. In another aspect, the vector is delivered using other delivery methods suitable for localized delivery, such as localized permeation of the blood-brain barrier. Particularly preferred delivery methods are those that deliver the vector to regions of the brain that require modification.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antigen, antibody or antibody portion) in the required amount in an appropriate solvent with one or a combination of ingredients enumerated above, as required, followed by filtered sterilization.

Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are generally known to those skilled in the art. The pharmaceutical compositions may include a “therapeutically effective amount” or a “prophylactically effective amount” of the vectors of the invention.

For therapy (e.g., of neurological disorders which may be ameliorated by a specific gene product) a therapeutically effective amount or dose of the vector expressing the therapeutic LAMuOR is administered to a subject in need of such treatment. The use of the vector disclosed herein in the manufacture of a medicament for providing therapy to a subject is within the scope of the present application.

A “therapeutically effective amount” refers to an amount effective, at dosages and for periods of time necessary, to achieve the desired therapeutic result. A therapeutically effective amount of the vector may vary according to factors such as the disease state, age, sex, and weight of the individual, and the ability of the vector to elicit a desired response in the individual. A therapeutically effective amount is also one in which any toxic or detrimental effects of the vector are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

In instances where human dosages for the vector have been established for at least some condition, those same dosages, or dosages that are between about 0.1% and 500%, more preferably between about 25% and 250% of the established human dosage can be used. Where no human dosage is established, as will be the case for newly discovered pharmaceutical compositions, a suitable human dosage can be inferred from ED50 or ID50 values, or other appropriate values derived from in vitro or in vivo studies, as qualified by toxicity studies and efficacy studies in animals.

A therapeutically effective amount of the vector can be administered to a subject at various points of time. For example, the vector can be administered to the subject prior to, during, or after the subject has developed a disease or disorder. The vector can also be administered to the subject prior to, during, or after the occurrence of a disease or disorder.

The dosing frequency of the vector can vary. For example, the vector can be administered to the subject once in a lifetime. In other aspect, the dosing can be about once every week, about once every two weeks, about once every month, about one every six months, about once every year, about once every two years, about once every three years, about once every four years, about once every five years, about once every six years, about once every seven years, about once every eight years, about once every nine years, about once every ten years, or about once every fifteen years. In some embodiments, the vector is administered to the subject at most about once every week, at most about once every two weeks, at most about once every month, at most about one every six months, at most about once every year, at most about once every two years, at most about once every three years, at most about once every four years, at most about once every five years, at most about once every six years, at most about once every seven years, at most about once every eight years, at most about once every nine years, at most about once every ten years, or at most about once every fifteen years.

The vectors disclosed herein can be administered to a subject (e.g., a human) in a method of treating opioid use disorder. In some embodiments of the methods of the disclosure, administering the composition reduces a symptom of OUD or prevents a relapse of the opioid use disorder in the subject.

Opioid use disorders are characterized by progressively uncontrollable opioid use that persists in spite of negative consequences (e.g., social, economic and/or medical consequences). Opioid use disorders are marked by a transition from opioid use that is well controlled, to a use that is unregulated and destructive. This transition can be abrupt or progressive in nature. Opioid use disorders are characterized by addiction, or dependence, upon the substance. When a subject is dependent upon, or addicted to, an opioid, this means that there is a physical, physiological or psychological reaction and/or interaction of the opioid and the subject, which results in the subject exhibiting or having a forced or compulsive use of the opioid without a recognized purpose or need for treating a disease. Rather, the purpose is that of achieving the desired effect, and/or avoiding withdrawal symptoms as defined hereinafter, which occur when the opioid is discontinued or the amount used is reduced. Opioid use disorders are sometimes referred to as “opioid abuse”, and the opioid or opioids to which the subject is addicted or dependent upon are “abused”.

As used herein, the term “treatment” refers to an intervention made in response to a disease, disorder or physiological condition manifested by a patient, particularly a patient suffering from one or more serotonin-related diseases. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

In some aspects of the methods of the disclosure, administering the composition reduces tolerance to an opioid of the opioid use disorder in the subject. In many cases subjects addicted to an opioid develop an increased tolerance to the opioid. Tolerance frequently leads to increased opioid use to attempt to compensate for the diminished effects of the opioid that can result from tolerance.

In some aspects, administering the composition reduces craving or dependence on an opioid of the opioid use disorder in the subject. Craving denotes a heightened desire for a substance. Craving is often coupled to a difficulty in experiencing a cue, such as an environmental cue, without incurring a high level of desire for the substance. Craving can also include repeated failures to resist the urge to use the opioid, and intolerable mental or physical states in the absence of opioid use.

In some embodiments, administering the composition improves adherence to a treatment for the opioid use disorder in the subject by reducing withdrawal symptoms and reducing cravings. In some embodiments, administering the composition reduces a preference for an opioid of the opioid use disorder or decreases liking for an opioid of the opioid use disorder in the subject. In some embodiments, administering the composition increases abstinence from an opioid of the opioid use disorder in the subject.

In some embodiments of the methods of the disclosure, the method further comprises a behavioral therapy. In some embodiments, the behavioral therapy comprises counseling, a contingency management system, a mindfulness based therapy, a cognitive-behavioral therapy, a digitally administered behavioral therapy or a virtual reality based behavioral therapy. In some embodiments, the counseling is in person or digitally administered. In some embodiments, the method reduces the amount behavioral therapy required compared behavioral therapy without the administration of the composition.

In some embodiments of the methods of the disclosure, the method further comprises an additional pharmacotherapy for opioid abuse. In some embodiments, the additional pharmacotherapy comprises a gradually reducing regimen, a substitution therapy or a medication assisted treatment. In some embodiments, the substitution therapy comprises methadone, buprenorphine, or Sublocade. In some embodiments, the medication assisted treatment comprises a MuOR agonist, for example a positive allosteric modulator of MuOR, mu-PAM, that binds the receptor and enhances the binding affinity and/or efficacy of the endogenous opioid. In some embodiments, the medication assisted treatment is lofexidine or clonidine. Other medications include gabapentin, pregabalin, buprenorphine, cannabinoids, naltrexone and ketamine.

In some embodiments of the methods of the disclosure, the composition is administered prior to the additional therapy. In some embodiments, the composition is administered at the same time as the additional therapy. In some embodiments, the composition is administered after the additional therapy.

The invention is further illustrated by the following non-limiting examples.

The following Materials & Methods were used in the Examples below.

Animals. All experiments were conducted using male and female B6/FVB F1 hybrid mice, all of which were fathered by DAT^{IREScre} male mice (Stock #006660, Jackson Laboratory), and similarly express Cre recombinase in dopaminergic cells of the ventral tegmental area (VTA). Animals underwent one stereotactic surgery involving either one set of bilateral injections of an experimental AAV in VTA, or a single unilateral injection of experimental AAV in VTA, followed by ipsilateral injection of an AAV encoding for the dopamine biosensor dLight1.2 and chronic implantation with an optical fiber in nucleus accumbens (NAc). All animals underwent 4 weeks of recovery after surgery before undergoing any experiments. At the end of all experiments, mice were sacrificed by transcardial perfusion and their brains were harvested for histological analysis.

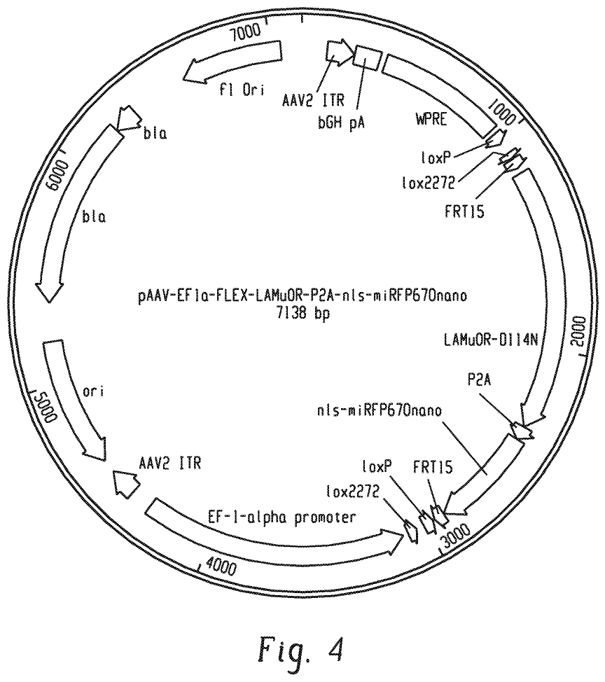

Viral Vectors. These experiments used AAV5-EF1a-FLEX-LAMuOR-P2A-nls-miRFP670nano (FIG. 4, AAV-LAMuOR, or active AAV), which contains Cre-dependent LAMuOR coexpressed with the near-infrared fluorescent marker nls-miRFP670nano (active AAV with LAMuOR-D114, FIG. 4). The control AAV contains the Cre-dependent nls-miRFP670nano alone as a negative control. The commercially available vector AAV5-hSyn-dlight1.2 (dLight1.2), a biosensor for extracellular dopamine binding, obtained through Addgene for use in fiber photometry recordings of dopamine release. Dose finding was executed by comparing groups of mice that receive different doses of experimental vector: active AAV (10⁹ vg, 10⁸ vg, or 10⁷ vg) or control AAV (10⁹ vg). Control AAV was added to the 10⁷ vg and 10⁸ vg AAV injection mixes so that all groups received the same total dose of AAV.

Rotation Assay. Mice unilaterally expressing active or control AAV in VTA were injected with saline or fentanyl and placed in an infrared-illuminated plastic square arena (30 cm×30 cm), where their movements were recorded on video for 30 min. On the first day, mice received a single i.p. injection of vehicle (0.9% saline, i.p.) before recording, and on day two, the experiment was repeated with a single i.p. injection of fentanyl (500 μg/kg, i.p.).

Fiber Photometry. Mice unilaterally express active or control AAV in the VTA. The dopamine biosensor dLight1.2 is expressed in the NAc, and an optical fiber was chronically implanted to measure the dLight signal, which reports dopamine concentration. During recordings, excitation (425 nm) and control LED light (560 nm) was passed through excitation filters and focused onto the patch cord mated to the animal's chronically implanted fiber, and emission light (500-550 nm and 580-680 nm) was collected and passed onto a photodetector. Once the fiber was mated, mice were placed in an infrared-illuminated plastic circular arena (15 cm in diameter), and baseline dLight1.2 signal was recorded for 10 min. Then each mouse received an IP injection of drug, and changes in dLight1.2 fluorescence were recorded for an additional 30 minutes.

Open Field Assay. Mice bilaterally expressing active or control AAV bilaterally in the VTA were injected with saline, escalating doses of fentanyl, or cocaine, and placed in an infrared-illuminated plastic square arena (30 cm×30 cm), where their movements were recorded on video for 30 min. Mice received a series of injections (0.9% saline vehicle, fentanyl 50 μg/kg, fentanyl 100 μg/kg, fentanyl 500 μg/kg, cocaine 20 mg/kg), spaced out over 13 days, with 2 days of rest in between each injection. Only one injection was received per session.

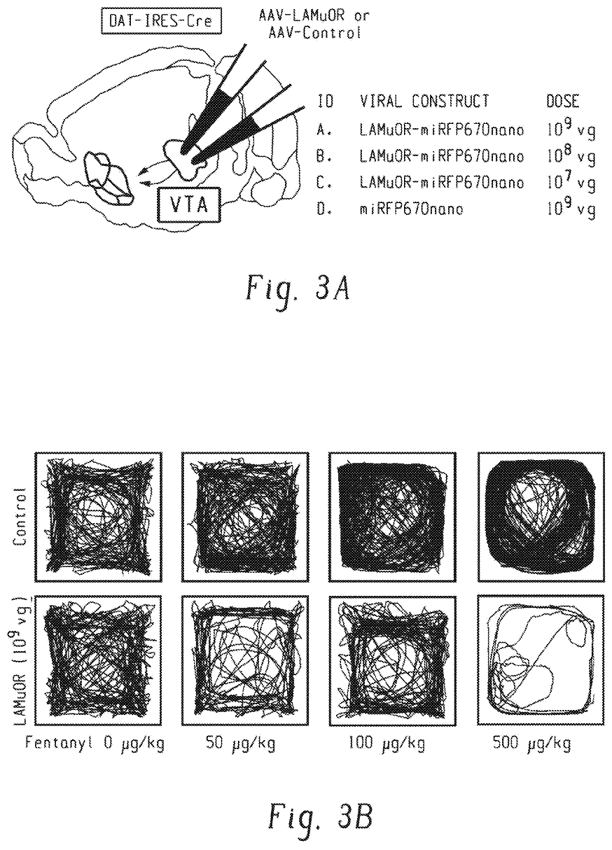

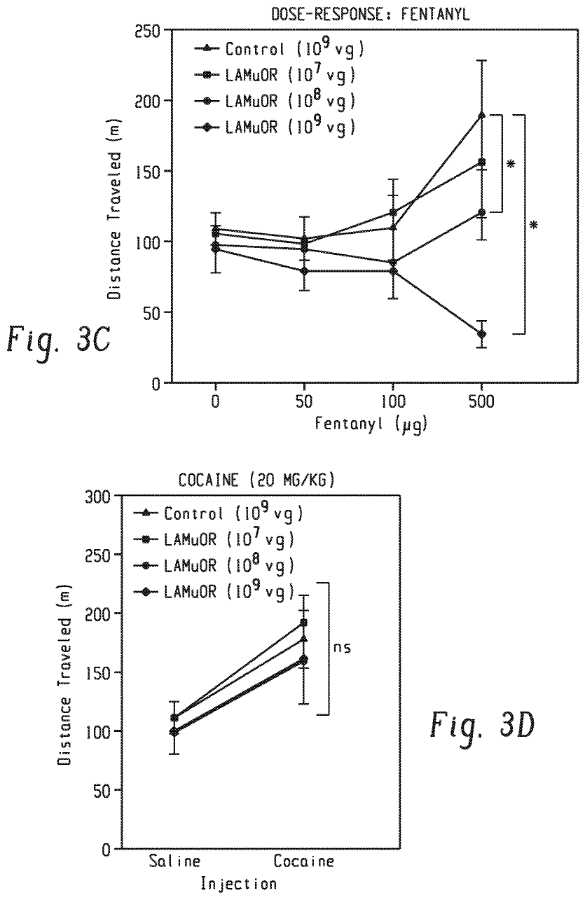

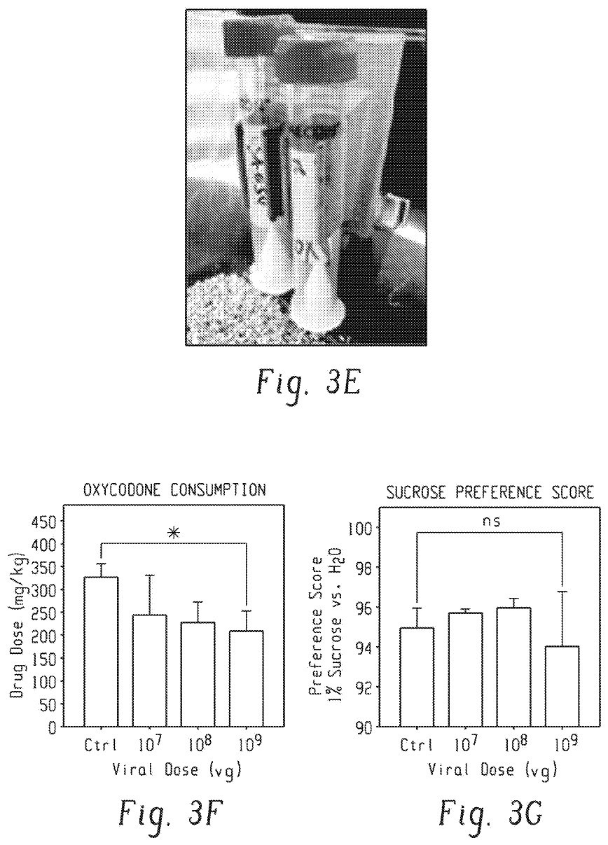

Oxycodone Two-Bottle Choice. To test the effects of LAMuOR on opioid consumption, mice were placed in clean cages containing two sipper bottles made out of conical vials and plastic sipper spouts (FIG. 3E). For the first 3 days, mice habituated to this new environment as both bottles were filled with tap water and switched positions every 24 hours. After habituation, a 5-day forced choice period was imposed by removing one of the drinking bottles and filling the other bottle with escalating doses of oxycodone solution. The oxycodone solution concentration started at 0.1 mg/ml on the first day, then increased to 0.3 mg/ml for the next two days, and increased again to 0.5 mg/ml on the last two days. The mice completed a 7-day free choice period as both bottles were made available in the cage again, with one bottle containing tap water, and the other containing 1.0 mg/ml oxycodone solution. Bottles were weighed and re-filled and switched positions every 24 hours, with each animal's oxycodone or water consumption recorded.

Sucrose Preference Test. To test the effects of LAMuOR on physiological rewards, mice were tested with the sucrose preference test, starting with habituation to two dual bearing sipper tubes, one containing plain water and the other containing a 1% sucrose solution, placed inside the home cage. After three days of habituation, sucrose and water intake were evaluated over an additional four days. The location of the sucrose and water tubes was switched every morning during both the habituation and evaluation periods. Sucrose preference scores were obtained by calculating the total volume of sucrose solution ingested/total volume of liquid ingested (H₂O+sucrose solution)×100%.

### Example 1: Opioid Use Disorder

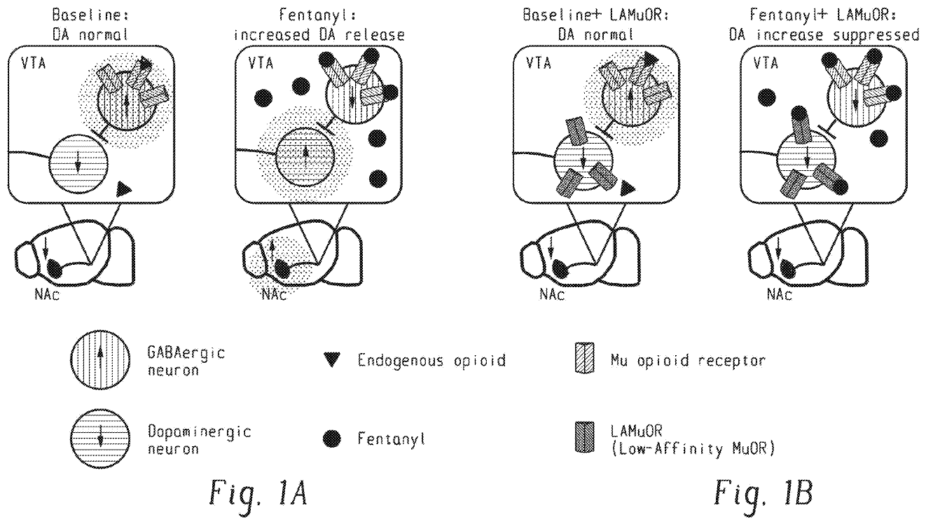

Opioid use disorder (OUD) has reached epidemic proportions in the United States, and although some effective treatments exist, it is a large and growing public health burden. The reinforcing effects of opioids are mediated by activation of the mu opioid receptor (MuOR), a Gi-coupled G-protein coupled receptor that exerts an overall inhibitory effect on MuOR-expressing cells. The MuOR is expressed in many cell populations throughout the brain, but a major contributor to the addictive potential of opioids is MuOR expression in GABAergic neurons of the ventral tegmental area (VTA, FIG. 1A). In the absence of opioids, these neurons inhibit the dopaminergic VTA neurons that project to the nucleus accumbens (NAc). Activation of these MuORs by opioids inhibits the GABAergic cells, thereby releasing the dopaminergic cells from inhibition and increasing dopamine levels in the nucleus accumbens. Increasing dopamine levels in the NAc causes euphoria and is believed to be the “final common pathway” of all forms of drug addiction, including OUD.

### Example 2: Design of LAMuOR (Low-Affinity Mu Opioid Receptor)

Finding a means to suppress or counteract this opioid-induced dopamine release could provide a powerful therapeutic strategy for OUD. However, MuOR antagonists paradoxically increase the risk of fatal overdose upon relapse, and systemic dopamine receptor antagonists would cause too many side effects to be a viable therapeutic strategy. One method for targeting the dopaminergic VTA cells with high specificity is chemogenetics, which refers to using gene therapy methods to express mutant neurotransmitter receptors in specific brain circuits. These receptors contain mutations that prevent them from being activated by endogenous ligands, and they are instead activated by specialized drug-like compounds designed to have minimal off-target effects. The most common chemogenetic receptors are called DREADDs (Designer Receptors Exclusively Activated by Designer Drugs), and the inhibitory DREADD hM4Di has been used successfully in mice to suppress VTA dopamine release, stopping heroin self-administration. However, DREADDs have two serious problems: 1) dopamine release would be suppressed all the time, not just in the presence of opioids. This would likely cause unacceptable neuropsychiatric side effects and affect physiological reward-related behaviors. 2) DREADDs will fail unless the patient adheres stringently to a daily medication regimen.

The rationale underlying LAMuOR (Low-Affinity Mu Opioid Receptor) as a tool to prevent opioid drugs from stimulating dopamine release in the NAc is shown in FIG. 1. Opioids of abuse, such as fentanyl or heroin, stimulate dopamine release indirectly by activating MuORs on GABAergic cells in the VTA (FIG. 1A). MuOR activation suppresses GABA release, which allows the VTA dopaminergic cells to become overactive and release supraphysiological amounts of dopamine in the NAc. Increased dopamine release in the NAc is believed to be the “final common pathway” of addictive drugs. FIG. 1B shows how the LAMuOR strategy counteracts this process by using adeno-associated viral vectors (AAVs) to express the human MuOR D116N mutant (shown and discussed as MuOR D114N mutant, or LAMuOR-D114N in the Examples and figures), ectopically in dopaminergic VTA cells. Under baseline conditions, the low affinity of the receptor prevents activation by endogenous opioids. However, the high concentrations of opioids present during opioid abuse activate LAMuOR and suppress DA release.

The novel chemogenetic approach described herein uses LAMuORs to specifically suppress opioid-induced VTA dopamine release. Our prototype is LAMuOR-D116N, a human MuOR containing the D116N mutation (equivalent to the rat D114N mutation exemplified herein), which preferentially reduces affinity for endogenous opioids but leaves affinity for exogenous opioids relatively intact. Like hM4Di, LAMuOR exerts an inhibitory effect through Gi activation, but it is activated by exogenous opioids rather than a traditional DREADD ligand. This avoids the problems associated with traditional DREADDs yet would confer a lifelong therapeutic effect from a single treatment, a distinct advantage over existing OUD treatments.

### Example 3: Mice Unilaterally Expressing LAMuOR in the VTA Demonstrate Strong Fentanyl-Induced Rotational Behavior

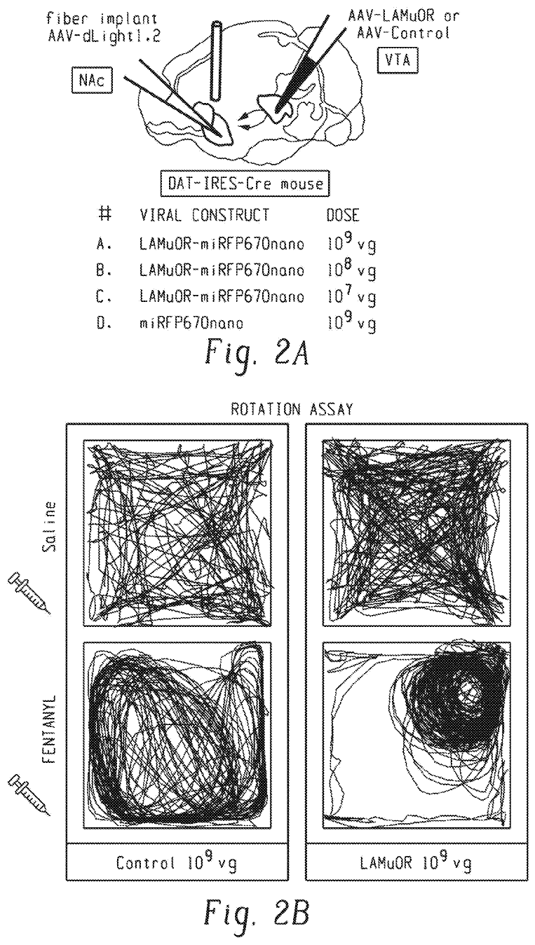

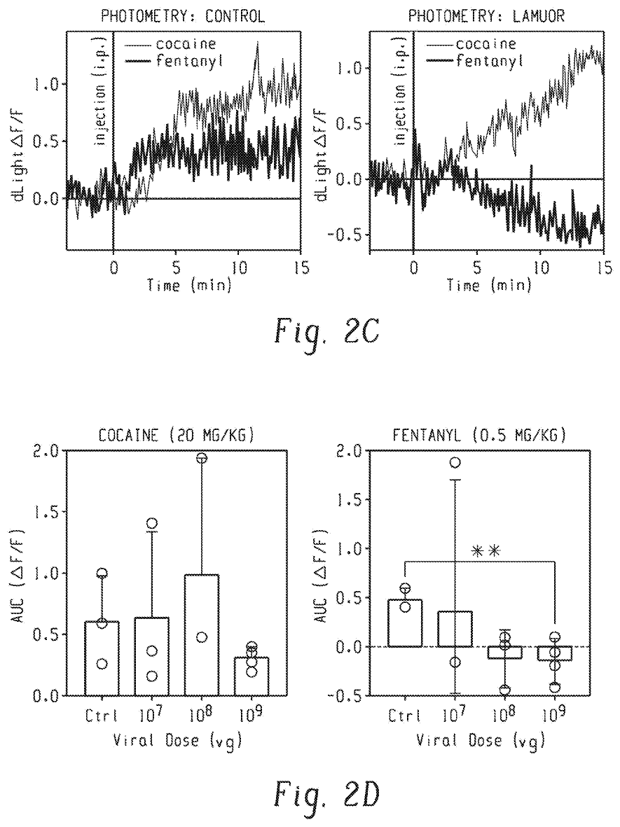

Rotation behavior is a well-established assay in which unilateral reductions in dopamine release relative to the opposite hemisphere lead to ipsiversive circling behavior. We used this test to determine whether LAMuOR's effects on dopamine release are able to affect behavior. Animals received unilateral injections of active or control AAV in VTA, and their behavior was recorded in the open field following injection with fentanyl (500 μg/kg, i.p.) or vehicle (0.9% saline, i.p.). LAMuOR-expressing animals exhibited marked rotation behavior in the direction ipsilateral to the AAV injection side, suggesting that NAc dopamine concentrations became lower on the LAMuOR expressing side relative to NAc of the opposite hemisphere in the presence of fentanyl (FIG. 2B).

### Example 4: Active AAV (AAV5-EF1a-FLEX-LAMuOR-P2A-nls-miRFP670nano—FIG. 4, LAMuOR-D114N-expressing AAV or AAV-LAMuOR) SUPPRESSES FENTANYL-BUT NOT COCAINE-INDUCED ELEVATIONS IN DOPAMINE CONCENTRATION IN NAc

To test the effects of LAMuOR on NAc dopamine release, fiber photometry recordings were performed on mice that received unilateral injections of active or control AAV in VTA and ipsilateral injections of AAV encoding the dopamine biosensor dLight1.2 followed by chronic fiber implantation in the NAc core. NAc dopamine levels were monitored following i.p. injection of either fentanyl or cocaine, with cocaine being used as a control condition to test if LAMuOR suppressed dopamine release in the absence of opioids. Group data expressed as the mean AUC of each trace after fentanyl injection show a significant reduction in mean dopamine release in mice that received AAV-LAMuOR 10⁹ vg compared to controls (p=0.006; Welch's t-test, t=4.838, df=4.56). **: p<0.01 (FIG. 2C, D).

### Example 5: LAMuOR Suppresses Fentanyl-Induced Hyperlocomotion, but not Baseline Locomotion or Cocaine-Induced Hyperlocomotion

Opioids are known to induce a hyperlocomotor response in mice. Because locomotion is modulated by the dopamine system, including by chemogenetic manipulation of VTA dopaminergic neurons, we evaluated LAMuOR's effects on fentanyl-induced hyperlocomotion. Total distance traveled was measured in the open field assay in response to increasing doses of fentanyl (50-500 μg/kg, i.p.), as well as to saline (negative control) and cocaine (20 mg/kg, i.p.), a positive control. Control mice show the expected escalation in activity with increasing doses of fentanyl (FIGS. 3B, 3C). Higher LAMuOR doses, however, demonstrate the opposite trend, with locomotion decreasing as the fentanyl doses increase, which a significant difference found between the control and LAMuOR 10⁹ animals at the 500 ug/kg dose (p=0.0159; unpaired t-test, Welsch's correction, t=7.180, df=2.127) (FIGS. 3B, 3C). As expected, LAMuOR-expressing mice show no change in drug-free or cocaine-induced hyperlocomotion (FIG. 3D).

### Example 6: LAMuOR Reduces Home-Cage Oxycodone Self-Administration

Recent research has demonstrated that chemogenetic inhibition of VTA dopamine cells can both prevent acquisition of heroin self-administration in mice. Given that LAMuOR engages the same Gi pathway, it is likely to have a similar effect on opioid drug self-administration when expressed in VTA dopamine cells. Following a recently published oxycodone two-bottle choice (2BC) paradigm, we have established a procedure in our lab for achieving oral oxycodone self-administration in mice (FIG. 3E). We used this 2BC task to test voluntary consumption of opioids in LAMuOR expressing mice. Mice were initially restricted to a single drinking bottle containing oxycodone at increasing doses (0.1-0.5 mg/ml, p.o.) over a forced-choice 5 day period, followed by a 7 day free choice period where mean oxycodone intake (1 mg/ml, p.o.) was compared with water intake. LAMuOR mice accumulated smaller mean doses of ingested oxycodone over the 14 day period compared to controls in an AAV-dose dependent manner, with LAMuOR 10⁹-expressing animals consuming the least (p=0.0224; unpaired t-test, Welch's correction, t=3.829, df=3.617) (FIG. 3F).

### Example 7: LAMuOR does not Alter Sucrose Preference

The VTA dopaminergic system serves a critical role in the modulation of physiological reward seeking and taking. Furthermore, the VTA contains endogenous opioid peptides that have the potential to interact with LAMuOR and affect opioid and DA-related physiological responses. To test whether LAMuOR would cause undesired effects on physiological reward processing, we compared how much water vs. 1% sucrose solution mice consumed in the Sucrose Preference Test and measured their preference as the mean total consumption of sucrose solution over a 4 day period divided by the total volume of liquid consumed after a 3 day habituation period. No differences in 4-day sucrose preference scores were found between any of the four experimental groups.

The particular instantiation of human LAMuOR-D114N (conventional rat numbering, equivalent to LAMuOR-D116N) that we have tested is packaged in an adeno-associated viral vector (AAV) that is activated by the presence of Cre recombinase (FIG. 4). We used this with transgenic mice that express Cre specifically in dopaminergic cells, providing us with targeting specificity. Subsequent versions for clinical use would not be activated by Cre, but instead would contain cis regulatory elements to target the cell population of interest. In our testing, we targeted VTA dopaminergic cells, but many other cell populations could also be targeted. Our AAV-LAMuOR-D114N construct is flanked by FRT15 sites, which enables it to be inactivated permanently by introduction of FlpO recombinase by a second AAV at a later date. This is a useful feature because it provides a means by which LAMuOR could be reversed if adverse effects were to occur.

Here we show that expressing LAMuOR in the VTA selectively reduces opioid-induced dopamine release in the NAc. This effect reduces the reinforcing properties of opioids, reducing fentanyl-induced hyperlocomotion and oxycodone consumption. Importantly, these effects are selective for exogenous opioids; responses to cocaine are intact, and sucrose preference is unaffected. These results provide an important proof of principle demonstration for LAMuOR as a potential therapeutic strategy to treat opioid use disorder.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of treating a subject with a substance use disorder, comprising administering to the subject a composition comprising a vector comprising an expression cassette, the expression cassette comprising a nucleic acid sequence encoding a low affinity human Mu opioid receptor (LAMuOR) expressible in a neuronal cell,
wherein, upon administering, the LAMuOR is expressed in DIR-positive direct pathway medium spiny neurons of the striatum of the subject in an amount effective to reduce direct pathway activity, and/or is expressed in dopaminergic neurons in the ventral tegmental area of the subject in an amount effective to reduce dopamine release, such that exogenous opioid-induced dopamine release is reduced without affecting release of endogenous opioid-induced dopamine,
wherein the administering reduces or prevents a relapse of the substance use disorder, reduces tolerance to or dependence on a substance of the substance abuse disorder, improves adherence of a treatment for a substance use disorder, reduces preference or decreases liking, or improves abstinence of a substance of the substance use disorder, in the subject.

2. The method of claim 1, wherein the substance use disorder is opioid use disorder.

3. The method of claim 1, wherein administering reduces opioid self-administration in the subject or suppresses or counteracts exogenous opioid-induced dopamine release in the subject or suppresses activation of direct pathway medium spiny neurons of the striatum.

4. The method of claim 1, wherein physiological reward-seeking behaviors of the subject are not affected.

5. The method of claim 1, wherein the method further comprises behavioral therapy comprising counseling, cognitive-behavioral therapy, a pharmacological therapy for substance abuse, including gradually reducing regimen, substitution therapy, or medication assisted treatment, or a combination thereof, and wherein the composition is administered prior to, at the same time as, or after the behavioral or pharmacological therapy, or a combination thereof.

6. The method of claim 1, wherein the opioid is fentanyl, heroin, mitragynine, codeine, hydrocodone, dihydrocodeinone, hydromorphone, meperidine, methadone, morphine, oxycodone, oxymorphone, remifentanil, carfentanil, sufentanil, buprenorphine, or a combination thereof.

7. The method of claim 1, wherein treatment is a single administration of the vector and is effective for the life of the patient.

8. The method of claim 1, wherein the nucleic acid encoding the LAMuOR contains a mutation at one or more amino acid position 116(2.50), 149(3.32), 331(7.46), 334(7.49) of the human mu Opioid receptor.

9. The method of claim 8, wherein the nucleic acid encoding the LAMuOR contains a D116N mutation and has a sequence identified in SEQ ID NO:1.

10. The method of claim 1, wherein the expression cassette further comprises Flp recombinase sites flanking the nucleic acid sequence encoding the LAMuOR, wherein, in the presence of a Flp recombinase the expression cassette is inactivated.

11. An expression cassette comprising a nucleic acid sequence encoding a low affinity Mu opioid receptor (LAMuOR) and an expression control element for expression in a neuronal cell.

12. The expression cassette of claim 11, wherein the nucleic acid encoding the LAMuOR contains a mutation at one or more amino acid position 116(2.50), 149(3.32), 331(7.46), 334(7.49), in its coding sequence.

13. The expression cassette of claim 12, wherein the mutation is a D116N mutation and the nucleic acid encoding the LAMuOR has a sequence identified in SEQ ID NO:1.

14. The expression cassette of claim 11, further comprising Flp recombinase sites flanking the nucleic acid encoding the LAMuOR, wherein, in the presence of a Flp recombinase the expression cassette is inactivated.

15. The expression cassette of claim 11, operably linked to an expression control element for driving expression of the LAMuOR nucleic acid in the neuronal cell.

16. The expression cassette of claim 11, wherein the neuronal cell is a dopaminergic neuron or a medium spiny neuron.

17. The expression cassette of claim 11, wherein the expression control element is a cytomegalovirus immediate early promoter/enhancer, a Rous sarcoma virus promoter/enhancer, an elongation factor 1 alpha promoter (EF1α), a neuron-specific promoter, or a combination thereof.

18. The expression cassette of claim 17, wherein the neuron specific promoter is a motor neuron promoter, a sensory neuron promoter, an interneuron promoter, a brain specific neuron in regions of the midbrain, the cortex, striatum, substantia nigra or hippocampus.

19. A vector comprising the expression cassette of claim 11.

20. A neuronal cell transformed with a vector of claim 19.
